(19) United States
(12) Patent Application Publication
(10) Pub. No.: US 2021/0169958 A1
(43) Pub. Date: Jun. 10, 2021

(54) Preparation Method of Agrocybe Aegerita Extract and its use in Preparing Medicine for Lowering Uric Acid

(71) Applicants: Guangdong Institute of Microbiology (Guang Dong Detection Center of Microbiology), Guangzhou (CN); Guangdong Yuewei Edible Fungi Technology Co., Ltd., Guangzhou (CN)

(72) Inventors: Tianqiao Yong, Guangzhou (CN); Yizhen Xie, Guangzhou (CN); Chunwei Jiao, Guangzhou (CN); Danling Liang, Guangzhou (CN)

(21) Appl. No.: 15/733,622

(22) PCT Filed: Aug. 14, 2018
(86) PCT No.: PCT/CN2018/100314

§ 371 Date: Sep. 17, 2020

(30) Foreign Application Priority Data
May 14, 2018 (CN) 201810456194.4

(51) Int. Cl. A61K 36/06; A61P 7/00

(52) U.S. Cl. CPC A61K 36/06; A61P 7/00; A61K 2236/15; A61K 2236/51; A61K 2236/39; A61K 2236/331

(57) ABSTRACT

The present invention relates to a preparation method of Agrocybe aegerita extract and its use in the preparation of lowering uric acid drugs, including: (1) Crush the dried fruit bodies of Agrocybe aegerita; (2) Separate the filter residue and the filtrate within 120 hours of extraction with 100-3000% ethanol by volume weight percentage at 0-100° C., and repeat it for 1-10 times; (3) Combine the filtrates obtained in step (2), concentrate to 25-35 mL and freeze-dry to obtain the alcohol extract; (4) Use 100-3000% water by volume percentage as a solvent to extract the alcohol-extracted filter residues of step (2) at 0-100° C. within 120 hours, separate the filter residue and the filtrate, repeat it for 1-10 times; (5) The filtrates obtained in step (4) are combined and concentrated to 25-35 mL, and lyophilized to obtain the water extract. The Agrocybe aegerita extract has a significant lowering uric acid effect with small side effects, can be used to prepare medicines for relieving gout symptoms, and provides a new direction for improving the current phenomenon of high side effects of gout drugs.

### TECHNICAL FIELD

The invention relates to the extract of edible fungi and its use in the preparation of medicines, in particular to a preparation method of Agrocybe aegerita extract and its use in the preparation of lowering uric acid medicines.

### BACKGROUND

With the changes in modern living habits and diet, the incidence of hyperuricemia has increased. The onset of hyperuricemia is due to increased production of uric acid and decreased excretion of uric acid in the kidneys, making the concentration of uric acid in the body higher than the blood dissolving capacity (>360 μmol/L). Uric acid is deposited in the joints in the form of crystals, and the repeated attacks cause related diseases such as gouty arthritis and uric acid kidney stones. These deposited crystals also cause recurrent severe inflammatory pain in soft tissues, joints and bone tissues, which seriously reduces the quality of life of patients.

The treatment of hyperuricemia requires lowering the blood uric acid concentration. Specific transporters on the cells of the nephron, such as renal uric acid transporter 1, are important targets for hyperuricemia. Opioid drugs, such as probenecid, sulpyridone and benzbromarone, are drugs that directly act on renal tubular targets, which increase renal excretion of uric acid by inhibiting urate, and reabsorb proteins by interacting with one or more transporters. Their use is limited due to their adverse side effects such as allergic reactions. Xanthine oxidase (XOD) is another important target, mainly distributed in the liver and intestines, and oxidizes hypoxanthine and xanthine to uric acid in the purine metabolism pathway. Lowering Uric acid drugs that inhibit XOD can be divided into purines and non-purines. Allopurinol is the most commonly used clinical XOD purine inhibitor, but it has been controversial due to toxic side effects such as Stevens-Johnson syndrome and nephrotoxicity. As a non-purine XOD inhibitor, Febuxostat can also cause cardiovascular complications, and the Food and Drug Administration (FDA) also supplemented the warning statement for this drug. Therefore, it is highly desirable to develop new, more effective and safe drugs for lowering high uric acid.

Edible mushroom Agrocybe aegeritais that is widely grown and commercially cultivated in Asia, North America and Europe. It is rich in protein, polysaccharides with hypoglycemic activity, indole derivatives, sesquiterpenes and alkaloids, in addition to sterols that inhibit the formation of osteoclasts and several antifungal and antibiotic compounds.

### SUMMARY OF THE INVENTION

In view of the above shortcomings, the present invention provides a method for preparing Agrocybe aegerita extract and its use in the preparation of lowering uric acid drugs, with ethanol and water as solvents which are resource-rich, inexpensive, and environmentally friendly to reduce production costs and avoid organic solvents pollution.

The present invention achieves the above objectives through the following solutions: In the first aspect, there is a preparation method of Agrocybe aegerita extract, including:

(1) Crush the dried fruit bodies of Agrocybe aegerita;

(2) Separate the filter residue and the filtrate within 120 hours of extraction with 100-3000% ethanol by volume weight percentage at 0-100° C., and repeat it for 1-10 times;

(3) Combine the filtrates obtained in step (2), concentrate to 25-35 mL and freeze-dry to obtain the alcohol extract;

(4) Use 100-3000% water by volume percentage as a solvent to extract the alcohol-extracted filter residues of step (2) at 0-100° C. within 120 hours, separate the filter residue and the filtrate, repeat it for 1-10 times;

(5) The filtrates obtained in step (4) are combined and concentrated to 25-35 mL, and lyophilized to obtain the water extract.

Preferably, the extraction temperature in the step (2) is 65° C.

Further preferably, the step (2) included extraction with ethanol at 65° C. for 3 hours.

Preferably, the extraction temperature in the step (4) is 85° C.

More preferably, the step (4) includes extracting the alcohol-extracted Agrocybe aegerita filter residues with water at 85° C. as a solvent for 3 hours.

Preferably, the extraction in the step (2) is ultrasonic extraction.

Preferably, the extraction in the step (4) is ultrasonic extraction.

Further preferably, the extraction in the steps (2) and (4) is ultrasonic extraction.

Preferably, the concentration in the step (2) is vacuum distillation concentration.

Preferably, the concentration in the step (4) is vacuum distillation concentration.

Preferably, the concentration in steps (2) and (4) is vacuum distillation concentration.

Preferably, the ethanol in the step (2) is 2% ethanol by volume weight percentage.

Preferably, the water in the step (4) is 100-3000% water by volume percentage.

In a preferred embodiment, there is a preparation method of Agrocybe aegerita extract, which includes:

(1) Crush the dried fruit bodies of Agrocybe aegerita;

(2) Extract with 300% ethanol by volume weight percent in a 65° C. water bath for 3 hours, then filter under reduced pressure to separate the filter residue and filtrate, and repeat it for three times;

(3) Combine the filtrate obtained in step (2) and concentrate it to 25-35 mL by distillation under reduced pressure, and freeze-dry to obtain the alcohol extract;

(4) Use 300% water by volume percent as a solvent to extract the filter residue obtained in step (2) in a water bath at 85° C. for 3 hours, then filter the residue and filtrate under reduced pressure to separate the residue and the filtrate, repeat it for three times;

(5) Combine the filtrate obtained in step (4) and concentrate it to 25-35 mL by distillation under reduced pressure, and freeze-dry to obtain the water extract.

The extraction rate of the alcohol extract of Agrocybe aegerita (AAE) in the present invention is 4.15% or more.

The extraction rate of the Agrocybe aegerita water extract (AAW) in the present invention is 7.13% or more.

In the preparation method of Agrocybe aegerita extract of the present invention, suitable temperature, solvent dosage, extraction time, etc. and their combinations are selected to greatly increase the yield of the extract.

In a second aspect, Agrocybe aegerita extract prepared by the above-mentioned preparation method is provided, which includes Agrocybe aegerita alcohol extract and/or Agrocybe aegerita water extract.

Preferably, the aforementioned Agrocybe aegerita extract includes Agrocybe aegerita alcoholic extract and/or Agrocybe aegerita water extract, and the content of ergosterol in the extract is 0.01-50 mg/g.

In a third aspect, a use of the above-mentioned Agrocybe aegerita extract in reducing uric acid and treating and/or preventing related diseases is provided.

Preferably, the reduction of uric acid and the treatment and/or prevention of related diseases include reducing blood uric acid levels, improving hyperuricemia, and improving gout symptoms.

Further preferably, the related diseases for reducing uric acid and treating and/or preventing related diseases include gouty arthritis and uric acid kidney stones.

Preferably, the Agrocybe aegerita extract includes Agrocybe aegerita alcohol extract and/or Agrocybe aegerita water extract.

In a fourth aspect, there is provided a use of the above-mentioned Agrocybe aegerita extract in the preparation of lowering uric acid drugs, drugs for treating and/or preventing related diseases and/or health products.

Preferably, the reduction of uric acid and the treatment and/or prevention of related diseases include reducing blood uric acid levels, improving hyperuricemia, and improving gout symptoms.

Further preferably, the related diseases for reducing uric acid and treating and/or preventing related diseases include gouty arthritis and uric acid kidney stones.

Preferably, the Agrocybe aegerita extract includes Agrocybe aegerita alcohol extract and/or Agrocybe aegerita water extract.

The lowering uric acid experiment in mice with the Agrocybe aegerita extract prepared by the method of the present invention showed that continuous gavage of Agrocybe aegerita extract for 7 days can reduce the blood uric acid concentration of hyperuric acid mice to lower than that of normal mice.

Experiments have confirmed that Agrocybe aegerita extract of the present invention has a good lowering uric acid effect. Pharmacological experiments verify that xanthine oxidase XOD may be its uric acid-lowering target in the uric acid-lowering effect of the extract of the present invention.

The beneficial effects of the present invention are:

1. The preparation process of Agrocybe aegerita extract of the present invention is simple and low in cost.

2. The present invention uses ethanol and water as the solvent to extract Agrocybe aegerita, there is no problem of organic solvent pollution or residue, and it is safe and environmentally friendly.

3. The Agrocybe aegerita extract of the present invention has a significant effect of lowering uric acid, has small side effects, and is non-toxic to the liver and kidney, which can be used to prepare medicines for relieving gout symptoms. It provides a new way to improve the current phenomenon of high side effects of gout drug.

### BRIEF DESCRIPTION OF THE DRAWINGS

FIG. 1 shows the serum uric acid levels of mice in each group in the experimental example.

FIG. 2 shows the urine uric acid levels of mice in each group in the experimental example.

FIG. 3 shows the serum urea nitrogen levels of mice in each group in the experimental example.

FIG. 4 shows the serum creatinine levels of mice in each group in the experimental example.

FIG. 5 shows the weight gain of mice in each group in the experimental example.

FIG. 6 shows the liver coefficients of mice in each group in the experimental example.

FIG. 7 shows the kidney coefficients of mice in each group in the experimental example.

FIG. 8 shows the spleen coefficients of mice in each group in the experimental example.

FIG. 9 shows the liver XOD activity of mice in each group in the experimental example.

FIG. 10 shows the use of ELISA to detect the expression of ion transporter in mouse kidney.

FIG. 11 shows the use of Western blot to detect the expression of ion transporter in the mouse kidney in an experimental example.

FIG. 12 shows the protein expression level of OAT1.

FIG. 13 shows the protein expression level of URAT1 in the experimental example.

### DETAILED DESCRIPTION OF THE DRAWINGS

The present invention will be further described below in conjunction with specific embodiments.

### Example 1

Preparation of Agrocybe aegerita Ethanol Extract

Take 100 g of Agrocybe aegerita fruit body, add to the conical flask after crushing with pulverizer, add 2 L ethanol, and extract the mixture in a water bath at 65° C. for 3 hours and then filter it with a 400 mesh filter. Repeat the extraction experiment three times. The resulting filtrate is merged, then distilling under reduced pressure and concentrating, freeze-dried to obtain 4.15 g of Agrocybe aegerita ethanolic extract (AAE) with a yield of 4.15%.

### Example 2

Preparation of Agrocybe aegerita Water Extract

The alcohol-extracted Agrocybe aegerita residue (the residue in Example 1) was extracted with 2 L water as a solvent in a water bath at 85° C. for 3 hours, and then filtered under reduced pressure to separate the residue and the filtrate. The extraction experiment was repeated three times, and the obtained filtrates were combined, and was concentrated by pressure distillation and lyophilized to obtain 7.13 g of water extract (AAW), with a yield of 7.13%.

### Example 3

Determination of Ergosterol in Agrocybe aegerita Extract

Using methanol at a flow rate of 0.8 ml/min as the mobile phase, a linear gradient elution was performed on a reverse phase chromatography column (Waters Atlantis T3 RP-C18, 5 μm, 250 mm×4.6 mm). Eluting with methanol:water=5:95 for 65 minutes, and then eluting with methanol:water=95:5 for 65 minutes. The detection wavelength is 260 nm, and the column temperature is maintained at 25° C. The loading volume is 20 μl. The ergosterol content of the alcohol extract was measured to be 0.48%; the ergosterol content of the water extract was measured to be 0.027%.

### Experimental Example 1

The Pharmacological Experiment of Agrocybe aegerita Extract

Experiment Method:

(1) 100 male SPF Kunming mice (20±2 g) were randomly divided into 10 groups: normal control group, hyperuricemia model group, allopurinol control group and benzbromarone control group, and high, middle, and low doses Agrocybe aegerita alcohol extract administration group and Agrocybe aegerita aqueous extract administration group. Except for the normal control group by intraperitoneal injection and intragastric administration of distilled water, the other groups were intraperitoneally injected with oxazine potassium salt at a dose of 100 mg/kg/d, and simultaneously intragastrically with a dose of 600 mg/kg/d hypoxanthine for modeling. Fasting for one hour before making the model without water, one hour after making the model, give allopurinol (5 mg/kg) and benzbromarone (7.8 mg/kg) intragastrically to allopurinol control group and benzbromarone control group. For the drug group, the low, middle, and high-dose Agrocybe aegerita alcohol extract administration group (Agrocybe aegerita alcohol extract, AAE) were respectively 50, 100, 200 mg/kg concentration of the Agrocybe aegerita alcohol extract prepared in Example 1 100 mg/kg/d for intragastric administration. Low, middle, and high-dose Agrocybe aegerita water extract administration group (Agrocybe aegerita water extract, AAW) were respectively 50, 100, 200 mg/kg concentration of the Agrocybe aegerita water extract prepared in Example 2 100 mg/kg/d for intragastric administration. The normal control group and the hyperuricemia model control group were gavage with the same volume of pure water for 7 consecutive days, and in the 1, 3, 5, and 7 days to weigh the mice.

(2) One hour after intragastric administration on the 7th day, the mice were sacrificed, and the blood and urine were collected by centrifugation at a speed of 3500 r/min for 10 min. The blood samples were separated and the serum was stored at −20° C. The organs of the mice were removed, including the liver, spleen, and kidneys, and washed with physiological saline solution. Then blot it dry with an ordinary filter and weigh it.

(3) Take the serum and urine obtained in step (2) to measure serum uric acid levels, urine uric acid levels, serum urea levels and creatinine levels, and urine urea levels and creatinine levels.

(4) Take the organs obtained in step (2) and calculate the organ coefficients (liver coefficient, spleen coefficient and kidney coefficient) by dividing the weight of a single mouse by the weight of each organ of a single mouse. The liver and kidney tissues were excised, weighed and homogenized with cold normal saline (0.9%), and centrifuged at 2,400 rpm for 10 minutes at 4° C. The supernatant was retained for XOD activity analysis and Western blot analysis.

(5) Determine the liver XOD activity of the liver tissue fluid obtained in step (4) by ELISA kit colorimetry.

(6) Analyze the expression of URAT1 in mouse kidney by ELISA kit.

(7) Western blot analysis of kidney samples. Using GAPDH (glyceraldehyde-3-phosphate dehydrogenase) as an internal control, the expression of proteins including URAT1 (urate transporter 1) and OAT1 (organic anion transporter 1) were detected.

(8) Statistical analysis is performed using the professional data processing program SPSS (Release 11.5, SPSS Inc., 2001). All data are expressed as mean±standard error (S.D.) and analyzed by one-way analysis of variance (ANOVA), and group means were compared by two-tailed Student's t test. Differences with statistical significance (P<0.05 or P<0.01) are indicated by the following symbols: There is a differences from the normal control group: *P<0.05, **P<0.01. There is a difference between the control and PO and HX-induced hyperuricemia model group, #P<0.05, ##P<0.01. There is a difference with the allopurinol group control, ΔP<0.05, ΔΔP<0.01.

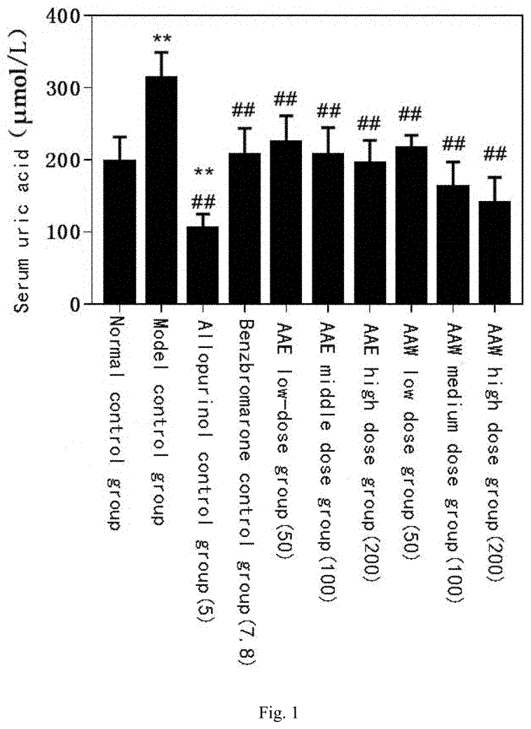

The results are shown in FIGS. 1-13.

Test Results:

Serum uric acid is a direct indicator to evaluate the effect of lowering uric acid. The results of serum uric acid levels of each group are shown in FIG. 1. Compared with normal mice (196 μmol/L), the hyperuricemia model group (313 μmol/L) induced by the combination of potassium oxazinate and hypoxanthine increased serum uric acid levels, which proved the success of modeling. Two positive drugs, allopurinol (5 mg/kg) and benzbromarone (7.8 mg/kg) reduced the blood uric acid of hyperuricemia mice to 105 and 206 μmol/, which also proved the success of modeling. The important fact is that AAE reduced blood uric acid in hyperuricemia mice to 223, 206 and 196 μmol/L at doses of 50, 100 and 200 mg/kg. AAW reduced the blood uric acid of hyperuricemia mice to 215, 162 and 140 μmol/1 at doses of 50, 100 and 200 mg/kg. The above results showed that the Agrocybe aegerita extract has good lowering uric acid efficacy.

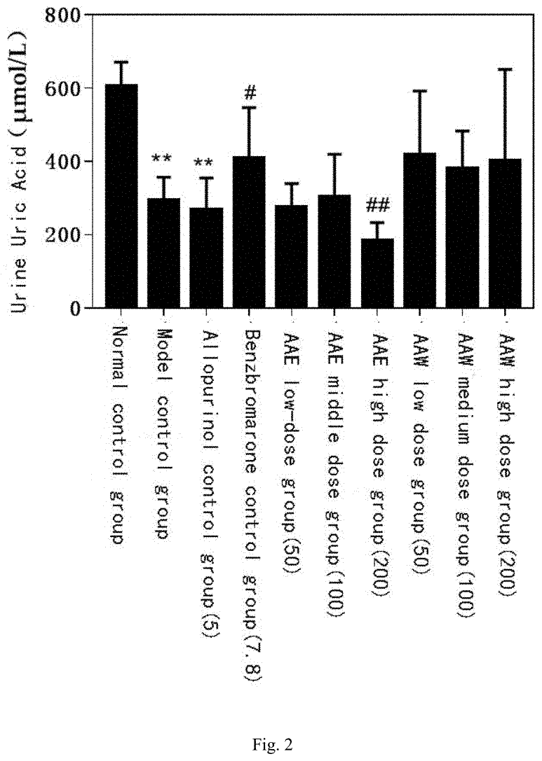

Since uric acid is excreted through the kidneys and is directly related to the blood uric acid level, in order to clarify whether the decrease in blood uric acid level treated with AAE and AAW is due to the enhancement of renal uric acid excretion or not, we determined their effect on the uric acid level, which showed in FIG. 2. Compared with the normal group (605 μmol/L), a decrease in the level of uric acid was observed in the model group (293 μmol/L, P<0.01). The administration of allopurinol (267 μmol/L) will further reduce the uric acid content. The uric acid content with benzbromarone at 7.8 mg/kg recovered to 408 μmol/L (P<0.05). The uric acid with AAE and AAW at doses of 50, 100, and 200 mg/kg were respectively 275, 303, 184, 418, 380, and 401 μmol/L, which did not significantly increase the content of uric acid. Therefore, the efficacy of Agrocybe aegerita for lowering uric acid may not be achieved by increasing renal uric acid excretion.

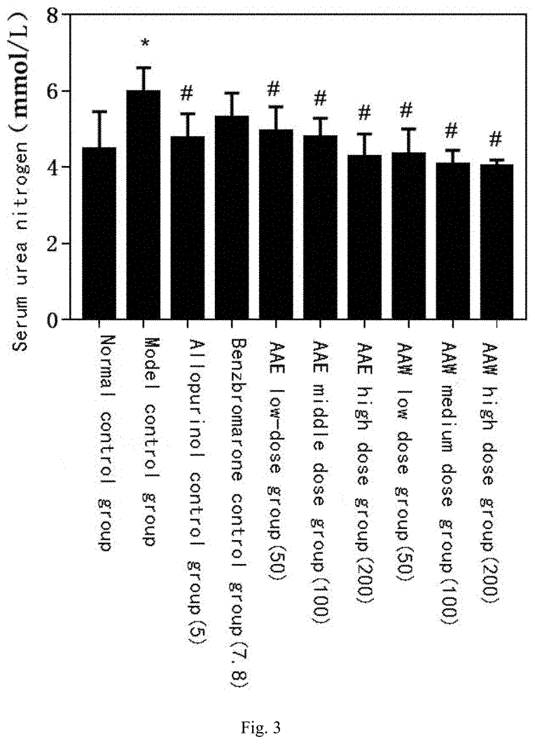

To study the effects of AAE and AAW on renal function, we also measured the renal function parameters of hyperuricemia mice. Urea nitrogen (BUN) is one of the main indicators of kidney function. Urea nitrogen is the main end product of human protein metabolism, which is filtered and excreted by the glomerulus (an important part of the kidney). In the early stage of renal damage, blood urea nitrogen can be in the normal range. When the glomerular filtration rate drops below 50% of normal, the concentration of blood urea nitrogen rises rapidly. Therefore, the blood urea nitrogen level reflects the status of kidney function as below: the higher the blood urea nitrogen level, the more serious damage to the kidney function. The serum urea nitrogen levels of each group were determined, and the results are shown in FIG. 3. The combined administration of potassium oxonate and hypoxanthine partially replaced the kidney injury effect of the pure potassium oxonate administration model, the hyperuric acid model group (5.96 mmol/L) had a higher BUN level than normal mice (4.47 mmol/L, P<0.05). The BUN (4.74 mmol/L, P<0.05) of the allopurinol group was lower than that of the model group, indicating that allopurinol impaired the renal function of hyperuricemia mice. Compared with the allopurinol group, administration with different doses of AAE and AAW, the AAE serum BUN parameters were respectively 4.92, 4.78 and 4.25 mmol/L, and the AAW group serum BUN parameters were respectively 4.32, 4.06 and 4.00 (P<0.05), which is significantly lower than that of the allopurinol control and the difference was statistically significant, and similar to the normal group. This shows that, compared with nephrotoxic allopurinol, Agrocybe aegerita extract has no nephrotoxicity.

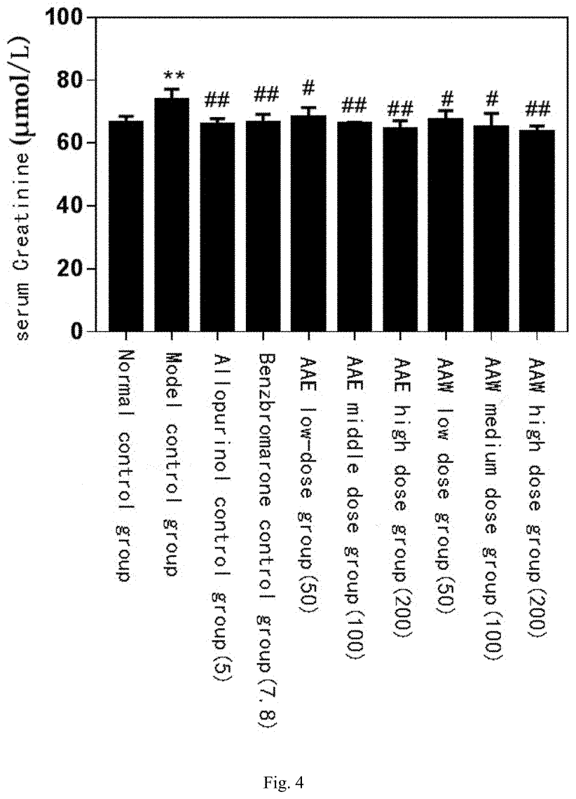

Creatinine is the metabolic end product of nitrogen-containing organic metabolites. It is filtered by the glomerulus and excreted in the urine. However, if kidney function is impaired, creatinine levels will rise. Therefore, the creatinine value has become one of the main indicators for evaluating renal function. The serum creatinine level of each group was measured. The results are shown in FIG. 4. The administration of potassium oxazine and hypoxanthine can make the serum creatinine level of normal mice (66.27 mmol/L) increased to (73.54 mmol/L, P<0.01). Compared with the hyperuricemia model group, the serum creatinine levels of the AAE-administered groups at doses of 30, 60, and 120 mg/kg respectively recovered to 68.10, 65.92, and 64.27 mmol/L. The serum creatinine levels of the AAW-administered groups at doses of 30, 60, and 120 mg/kg respectively recovered to 67.15, 64.75 and 63.37 mmol/L, indicating that the Agrocybe aegerita extract of Example 1 of the present invention has a certain effect on the recovery of kidney organs and has a certain degree of kidney protection.

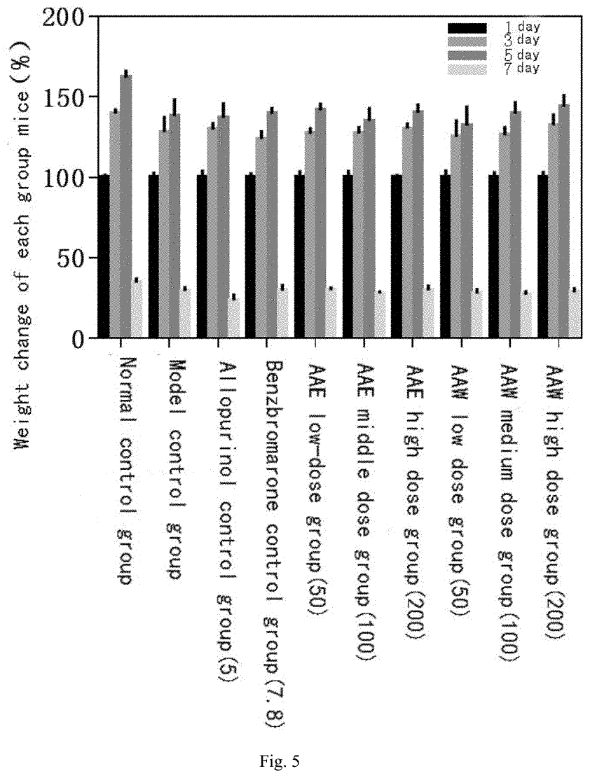

The body weight changes of mice in each group are shown in FIG. 5. Compared with mice in the normal group, all doses of AAE and AAW used in this study did not affect body weight (P>0.05). Allopurinol inhibited the weight increase of mice and it was toxic. This shows that Agrocybe aegerita extract is generally not toxic.

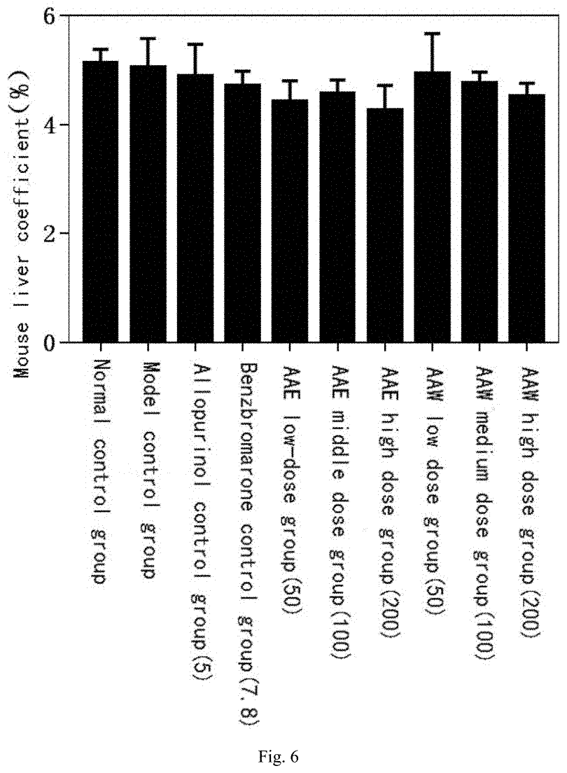

The weight change of internal organs is a sensitive indicator. In toxicology experiments, the organ coefficient between the administered group and the Non-administered group is usually used to evaluate the toxicity of the drug. The liver coefficients of each group of mice are shown in FIG. 6. There is no difference in liver coefficients in all treatment groups, indicating that allopurinol, AAE and AAW have little effect on liver function.

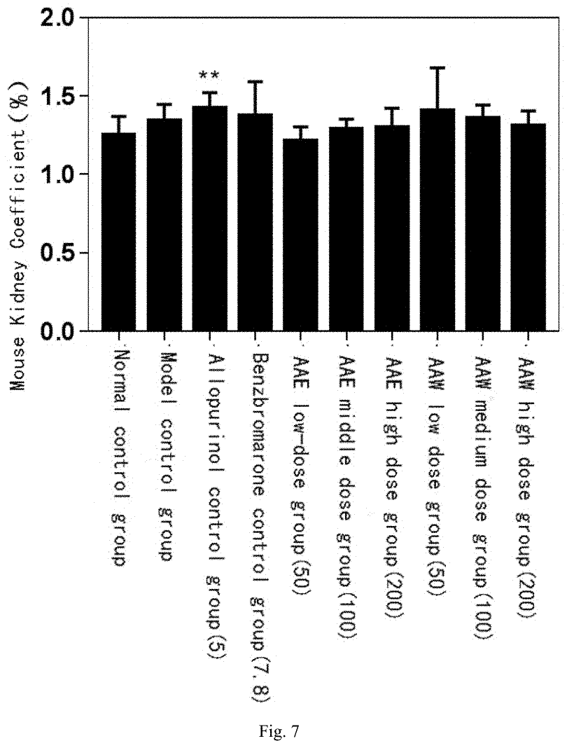

The renal coefficient of each group of mice is shown in FIG. 7. The renal coefficient of the allopurinol group (1.42%) was significantly higher than that of normal (1.25%, P<0.01). It shows that allopurinol has a toxicological effect on the kidney. In addition, vesicular nephritis was observed in the allopurinol group, but not in other groups. This indicates that potassium oxazine and allopurinol have certain damages to renal function. The renal coefficients of AAE at different doses were 1.21%, 1.28%, and 1.29%, and the renal coefficients of AAW at different doses were respectively 1.40%, 1.36%, and 1.31%. Compared with the normal group, the difference was not statistically significant, indicating that the AAE and AAW extracts of Agrocybe aegerita of the embodiment of the present invention have no or little effect on kidney function.

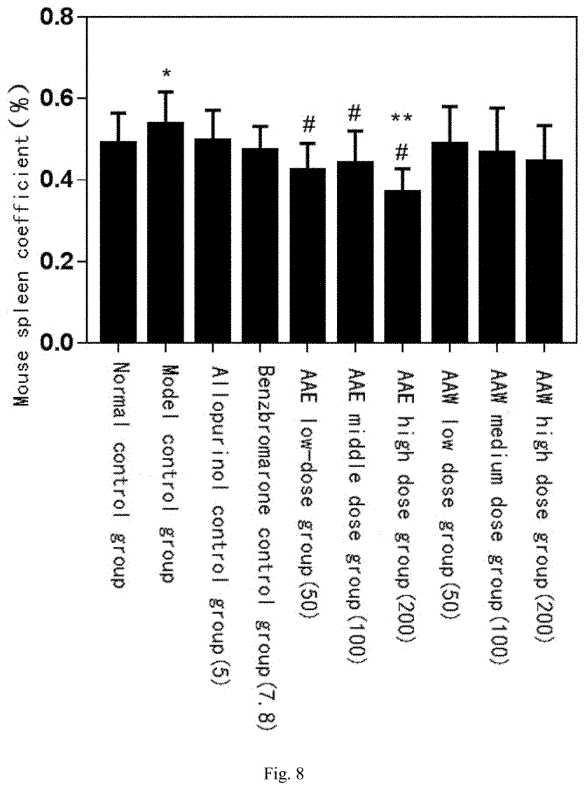

The spleen coefficient of mice in each group is shown in FIG. 8. The spleen coefficient of the model group (0.53%) is slightly higher than that of the normal group (0.49%, P<0.05). AAE showed lower spleen coefficients of 0.42, 0.44, and 0.37% (P<0.05). There was no significant difference in spleen coefficients among AAW groups.

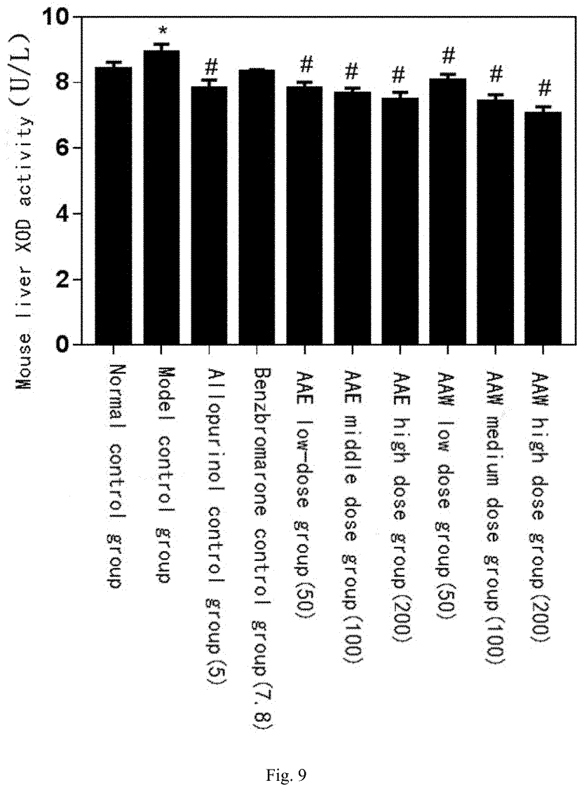

XOD (Xanthione oxidase) directly regulates the level of uric acid in vivo. Non-purine precursor substances undergo a series of biochemical transformations in vivo to produce purine nucleotides, which continue to decompose to form hypoxanthine and xanthine, and finally undergo continuous oxidation by XOD to form uric acid. It is mainly distributed in the liver and small intestine, and XOD activity is enhanced under hyperuricemia. The results of liver XOD activity are shown in FIG. 9. The liver XOD activity of the model group (8.91 U/L, P<0.05) is higher than that of the normal group (8.38 U/L). The positive control with a dose of 5 mg/kg of allopurinol significantly inhibited the liver XOD activity of hyperuricemia mice by 7.81 U/L (P<0.05). The XOD activities of AAE and AAW at doses of 50, 100, and 200 mg/kg were respectively 7.81, 7.64, 7.45, 8.06, 7.40 and 7.02 U/L, which significantly reduced liver XOD activity (P<0.05). These results may indicate that the uric acid-lowering effect of the AAE and AAW extracts of Agrocybe aegerita in the examples of the present invention may be through the inhibition of XOD activity.

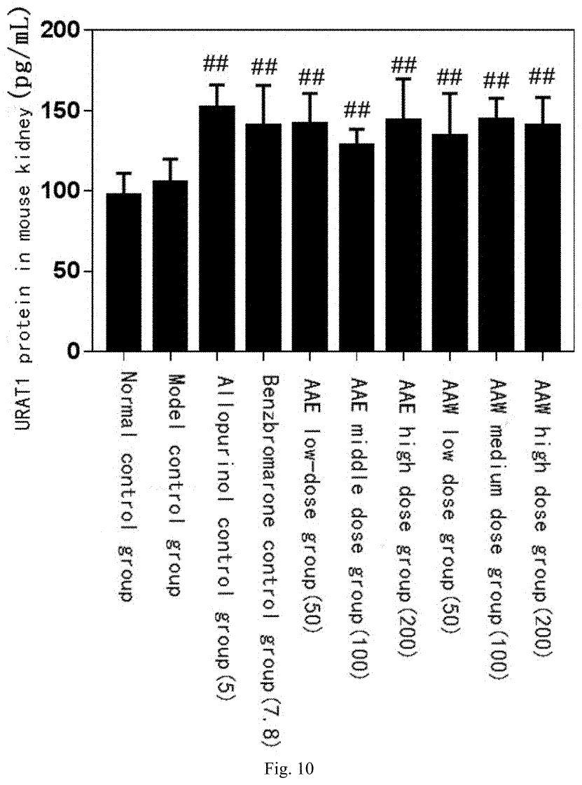

URAT1 (renal uric acid transporter 1) is the main target of uric acid excretion, which affects the reabsorption process of uric acid. Inhibiting URAT1 can promote uric acid excretion. As shown in FIG. 10, the expression level of the normal group (96.95 pg/mL) and the model group (104.85 pg/mL) was not significantly different, while the URAT1 protein expression level of the allopurine group was significantly increased (151.96 pg/mL, P<0.01). The URAT1 protein expression levels of low, middle and high doses of AAE and AAW were also higher than those of the model group, which were respectively 141.50, 128.12, 143.67, 133.96, 144.49 and 140.17 pg/mL. This may indicate that the Agrocybe aegerita extract of the present invention does not reduce the blood uric acid content by inhibiting URAT1.

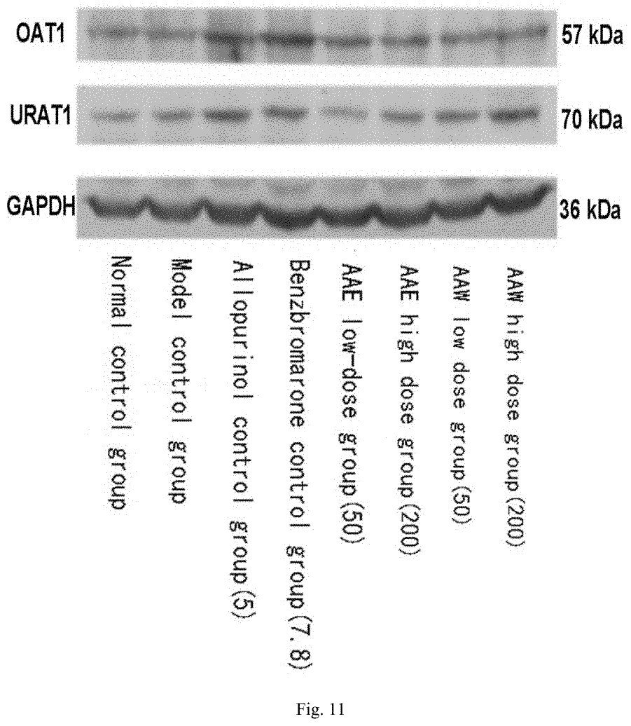

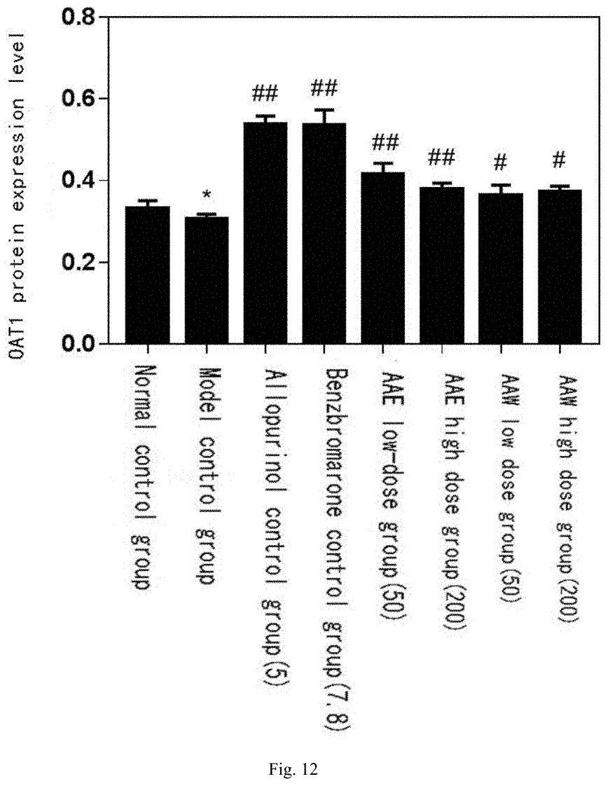

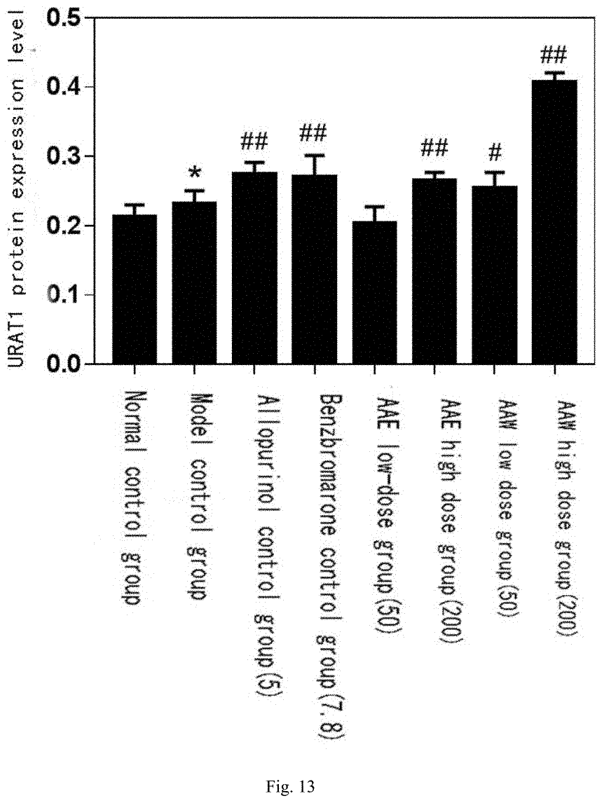

In addition, we tested the effect of Agrocybe aegerita extract on the protein levels of OAT1 and URAT1 in the kidney by western blotting (Western blot analysis), and the results are shown in FIG. 11. The gray analysis results of the protein expression level of OAT1 are shown in FIG. 12. The protein expression of OAT1 in the model group was lower than that in the normal group (P<0.05). Compared with the model group, each dose group of AAE and benzbromarone group can increase the expression of OAT1 (P<0.01). The expression of OAT1 protein in each group of AAW also increased significantly (P<0.05). The gray scale analysis results of the protein expression level of URAT1 are shown in FIG. 13. Compared with normal mice, hyperuricemia mice induced by potassium oxazine and hypoxanthine increased renal URAT1 levels (P<0.05), neither AAE nor AAW showed the effect of inhibiting URAT1 protein expression.

The above results indicate that the Agrocybe aegerita ethanol extracts (AAE) and water extracts (AAW) of the present invention significantly reduce the serum uric acid level of hyperuricemia mice, which is close to the normal group level. AAE and AAW showed inhibition of XOD activity but not URAT1 protein. It is also possible to reduce uric acid by increasing the protein expression of OAT1. In addition, compared with the existing clinical drugs with strong side effects, the Agrocybe aegerita extract has no toxicity to the liver and kidney, and can be used in the preparation of lowering uric acid and gout-improving drugs, health care products or auxiliary drugs.

The above are only preferred specific embodiments of the present invention, but the protection scope of the present invention is not limited thereto. Anyone familiar with the technical field within the technical scope disclosed in the present invention, according to the technology of the present invention, the equivalent replacement or change of the scheme and its conception shall be covered by the protection scope of the present invention.

1. The preparation method of Agrocybe aegerita extract, characterized in that it comprises:
(1) Crush the dried fruit bodies of Agrocybe aegerita;
(2) Separate the filter residue and the filtrate within 120 hours of extraction with 100-3000% ethanol by volume weight percentage at 0-100° C., and repeat it for 1-10 times;
(3) Combine the filtrates obtained in step (2), concentrate to 25-35 mL and freeze-dry to obtain the alcohol extract;
(4) Use 100-3000% water by volume percentage as a solvent to extract the alcohol-extracted filter residues of step (2) at 0-100° C. within 120 hours, separate the filter residue and the filtrate, repeat it for 1-10 times;
(5) The filtrates obtained in step (4) are combined and concentrated to 25-35 mL, and lyophilized to obtain the water extract.

2. The preparation method of Agrocybe aegerita extract according to claim 1, wherein the extraction temperature of the step (2) is 65° C.

3. The preparation method of Agrocybe aegerita extract according to claim 1, wherein the extraction temperature in the step (4) is 85° C.

4. The preparation method of Agrocybe aegerita extract according to claim 1, wherein the extraction in step (2) adopts ultrasonic extraction; and/or, the extraction in step (4) adopts ultrasonic extraction.

5. The preparation method of Agrocybe aegerita extract according to claim 1, wherein the concentration in step (2) is vacuum distillation concentration; and/or, the concentration in step (4) is vacuum distillation concentration.

6. The preparation method of Agrocybe aegerita extract according to claim 1, characterized in that the ethanol in the step (2) is 100-3000% ethanol by volume weight percentage; and/or, the water in the step (4) is 100-3000% water by volume weight percentage.

7. The preparation method of Agrocybe aegerita extract according to claim 1, characterized in that it comprises:
(1) Crush the dried fruit bodies of Agrocybe aegerita;
(2) Extract with 300% ethanol by volume weight percentage in a 65° C. water bath for 3 hours, then filter under reduced pressure to separate the filter residue and filtrate, and repeat it for three times;
(3) Combine the filtrate obtained in step (2) and concentrate it to 25-35 mL by distillation under reduced pressure, and freeze-dry to obtain the alcohol extract;
(4) Use 300% water by volume percentage as a solvent to extract the filter residue obtained in step (2) in a water bath at 85° C. for 3 hours, then filter the residue and filtrate under reduced pressure to separate the residue and the filtrate, repeat it for three times;
(5) Combine the filtrate obtained in step (4) and concentrate it to 25-35 mL by distillation under reduced pressure, and freeze-dry to obtain the water extract.

8-10. (canceled)

11. The preparation method of Agrocybe aegerita extract according to claim 2, wherein the step (2) is extracting with ethanol at 65° C. for 3 hours.

12. The preparation method of Agrocybe aegerita extract according to claim 3, wherein the step (4) is to extract the residue of Agrocybe aegerita after alcohol extraction with water as a solvent at 85° C. for 3 hours.

13. Agrocybe aegerita extract prepared by the preparation method of any one of claim 1-7 or 11-12, comprising Agrocybe aegerita alcohol extract and/or Agrocybe aegerita water extract; preferably, the ergosterol content in the extract is 0.01-50 mg/g.

14. The use of Agrocybe aegerita extract of claim 13 in the preparation of lowering uric acid drugs, drugs and/or health products for treating and/or preventing related diseases.

15. The use according to claim 14, wherein the reducing uric acid and treating and/or preventing related diseases include reducing blood uric acid levels, improving hyperuricemia, and improving gout symptoms; preferably, the reducing uric acid and treatment and/or prevention of related diseases include gouty arthritis and uric acid kidney stones.
